SEQUENCE LISTING (4 sequences)

SEQ ID NO: 1 - DNA, 1893 bp, Fagopyrum esculentum
atgacgtcactcacaatcacaagtagcttgccagcagtgagcaccgtagcaggagccacgtcttcaagcaaggcaatgttcttgcaaaatagtttcaaaaacactgcagggctccgacctctcaacgacgtcgatatgctcaaatgcagggccaactcaaataacgcaccttccaagttaccaacaatggaacccaagagatccgcaaataattctgtggaatcaagagttatccgatgtgggatgaacttggtctttgtaggagctgaggtagctccatggagcaaaaccggtggccttggagacgttcttgccggcctcccgcctgcaatggcggcgaatgggcaccgagtgatgacagtctccccgcgttatgatcagtaccaagacgcttgggacacaagcgtgcaagctgaggtaaaagtgggagataagacggaaactgtgcgattttttcactcttataagcgcggggttgatcgcgtctttgttgatcatccgttgttcttagccaaggtatggggaaaaactggattcaaaatctatggtccaagagccggcgaagattacgaagacaaccaacttcggtttagcctcttagcacaggcagctttggaagcaccaaggattcttaatctcagcaacaagttcttttcaggaccttatggtgaggatgttgtatttatcgccaatgattggcacactgcactgctgccatgctatctgaaaaccatgtacaaatcgagaggaatatacaacaatgcaaaggtggcgttttgcattcacaatatctcttaccaaggacgatttgcgcgatctgactacaattctctcaatttgcccgaagaattacttagatctttcaaatttcaagacgggtatgatacgccggtggaagggcctaagatcaactggatgcaagcaggaatactcgaatccgacaaggttgtgactgtgagcccgtattatgcggaagagctgatttcgggtgcagaaagaggagtcgagttagacactgtgattcgatcaaccggaattacagggatcttgaatggcatggacaaacaagagtggaatcctttaactgataaacacatcacttatcaatatgatgtttcaaatgtgatggaggcaaaggctttgaacaaggaagccttgcaagctgaggtgggattgcctgttgataggaacatacctcttataggattcattggaaggctagaggagcaaaagggttcggatattctcgtcgaatcaattcctcaatgcatcaatgagaacgtccagcttcttgttttgggaaccggaaagaagaaattcgagaaagaaatcgaagaactcgaaaatctgtatccaaacaaggttagaggagtgaccaagtttaacccagccctagctcacaagatcattgcaggggctgatttcatgctcataccaagccgattcgagccatgcgggcttatccaactacatgccatgctttatggaacggttcctatgtgtgcctcaactggaggacttgttgactgcgtcaaagaaggcattacaggattccacatgggccgattcggagcccagtgcaaagctgtagatgcggctgatgttgcagctgtgacatcgactgtgaggagggcagttgcagtgcatggtacacctgctatgagagacatgattctcaattgcatgtatcaagacctttcatggaaggcaagagcctgcaaggaaatgggaagagatgctacttggaatgcaggtggctgggagcgggcctgggatcaagggcgaagaaattgcgccgttagctgcggaaaacatcgccactccataatcaggagggttcatgtacctgcagaacagtataaggtgtatgactgtatgtcattactatcatga

SEQ ID NO: 2 - DNA, 1893 bp, Fagopyrum esculentum
atgacgtcactcacaatcacaagtagcttgccagcagtgagcaccgtagcaggagccacgtcttcaagcaaggcaatgttcttgcaaaatagtttcaaaaacactgcagggctccgacctctcaacgacgtcgatatgctcaaatgcagggccaactcaaataacgcaccttccaagttaccaacaatggaacccaagagatccgcaaataattctgtggaatcaagagttatccgatgtgggatgaacttggtctttgtaggagctgaggtagctccatggagcaaaaccggtggccttggagacgttcttgccggcctcccgcctgcaatggcggcgaatgggcaccgagtgatgacagtctccccgcgttatgatcagtaccaagacgcttgggacacaagcgtgcaagctgaggtaaaagtgggagataagacggaaactgtgcgattttttcactcttataagcgcggggttgatcgcgtctttgttgatcatccgttgttcttagccaaggtatgaggaaaaactggattcaaaatctatggtccaagagccggcgaagattacgaagacaaccaacttcggtttagcctcttagcacaggcagctttggaagcaccaaggattcttaatctcagcaacaagttcttttcaggaccttatggtgaggatgttgtatttatcgccaatgattggcacactgcactgctgccatgctatctgaaaaccatgtacaaatcgagaggaatatacaacaatgcaaaggtggcgttttgcattcacaatatctcttaccaaggacgatttgcgcgatctgactacaattctctcaatttgcccgaagaattacttagatctttcaaatttcaagacgggtatgatacgccggtggaagggcctaagatcaactggatgcaagcaggaatactcgaatccgacaaggttgtgactgtgagcccgtattatgcggaagagctgatttcgggtgcagaaagaggagtcgagttagacactgtgattcgatcaaccggaattacagggatcttgaatggcatggacaaacaagagtggaatcctttaactgataaacacatcacttatcaatatgatgtttcaaatgtgatggaggcaaaggctttgaacaaggaagccttgcaagctgaggtgggattgcctgttgataggaacatacctcttataggattcattggaaggctagaggagcaaaagggttcggatattctcgtcgaatcaattcctcaatgcatcaatgagaacgtccagcttcttgttttgggaaccggaaagaagaaattcgagaaagaaatcgaagaactcgaaaatctgtatccaaacaaggttagaggagtgaccaagtttaacccagccctagctcacaagatcattgcaggggctgatttcatgctcataccaagccgattcgagccatgcgggcttatccaactacatgccatgctttatggaacggttcctatgtgtgcctcaactggaggacttgttgactgcgtcaaagaaggcattacaggattccacatgggccgattcggagcccagtgcaaagctgtagatgcggctgatgttgcagctgtgacatcgactgtgaggagggcagttgcagtgcatggtacacctgctatgagagacatgattctcaattgcatgtatcaagacctttcatggaaggcaagagcctgcaaggaaatgggaagagatgctacttggaatgcaggtggctgggagcgggcctgggatcaagggcgaagaaattgcgccgttagctgcggaaaacatcgccactccataatcaggagggttcatgtacctgcagaacagtataaggtgtatgactgtatgtcattactatcatga

SEQ ID NO: 3 - DNA, 1686 bp, Fagopyrum esculentum
atggcgtctctaaataccgctgtgtgtatggcaagcagcggaggaaccacaactggctctgatgctagcaaggttgtgtcgttgcaaaatggtttaagaaaccacacggggcttcgtcctctcaacagagttgacatgctgaaatggagagccaacacttccaaattaccgagaatggaagagaagagagttgttacgagtcccagctttaaaaaaggggttattcagtgtggaatgaacttggtctttgtctcagctgaggtggctccttggagcaaaactggtggccttggggatgtccttgctggcctcccaccagccatggcggccaatgggcaccgagtcatgacaatctcccctcggtatgatcagtaccaagatgcatgggatacaagcgtggaggctgaggtaaaagtgggagataagacggaaactgtgcgctttttccactcttacaaacgtggggttgatcgtgtcttcgtggatcatccattgttcctagctaaggtatgggggaaaactggattccaaatctatggtccaagagctggtgaagattacgaagacaaccaacttcgattcagtgtcttatctcaggcagcattggaggctccaaggattctaaatctcaacaacaaatttttctcaggaccatatggagaggatgttttgtttgtcgcaaacgattggcactctgctctcctaccatgctacctgaagtccatgtacaaatcaaggggaatctataagaatgcaaaggttgcattctgcatccacaacatttcttatcaaggccgatttgccccaactgacttcaaagctctcaatttacccaatgaatttctgaagtcgtttgaattttttgatgggtatgacacgcctatcacaggggacaaaatcaactggatgcaagccggaatactagagtccgacaaggttgtgaccgtgagcccatattatgctgaagagttggtttcgggtgctgaaagaggtgtggaactagaaaaaatcattcgaaaaactggagtaattggtatcctaaatggaatggataaacaagagtgggatccttccagtgacaagttcatcaattacaaatatgatgtttcaactgtgatggaggcaaagccactcataaaggaatcccttcaagccgagttgggcttgcccgttgacaggaacattccacttataggattcattggaaggttggaagagcaaaagggttcggatattcttgttgaatccatcccagaatttatcaatgagaatgtccaaattgttatcttggggactggaaagaagcgatttgagaaacaaatcgaagagctcgaggccctttatcccgacaaggctagaggagtagcagcattcaacccatccatggctcacaagatcaatgctggtgttgactttatgttaatccctagtcggtttgagccatgtgggcttacccaactacacgctatgctctatggaacggttccaatttgtgcttcaactggtgggcttgtggattgtgtaaaagaaggcgtcacagggtttcacatgggcaagttcagcgccgaggagccagcaaagaaatgggaggagatgctactagggttacaggtggcggggagcggacctgggattgagggggaagaaatcgccccgcttgcagtggaaaatgttgctgctccttga

SEQ ID NO: 4 - DNA, 1686 bp, Fagopyrum esculentum
atggcgtctctaaataccgctgtgtgtatggcaagcagcggaggaaccacaactggctctgatgctagcaaggttgtgtcgttgcaaaatggtttaagaaaccacacggggcttcgtcctctcaacagagttgacatgctgaaatggagagccaacacttccaaattaccgagaatggaagagaagagagttgttacgagtcccagctttaaaaaaggggttattcagtgtggaatgaacttggtctttgtctcagctgaggtggctccttggagcaaaactggtggccttggggatgtccttgctggcctcccaccagccatggcggccaatgggcaccgagtcatgacaatctcccctcggtatgatcagtaccaagatgcatgggatacaagcgtggaggctgaggtaaaagtgggagataagacggaaactgtgcgctttttccactcttacaaacgtggggttgatcgtgtcttcgtggatcatccattgttcctagctaaggtatgagggaaaactggattccaaatctatggtccaagagctggtgaagattacgaagacaaccaacttcgattcagtgtcttatctcaggcagcattggaggctccaaggattctaaatctcaacaacaaatttttctcaggaccatatggagaggatgttttgtttgtcgcaaacgattggcactctgctctcctaccatgctacctgaagtccatgtacaaatcaaggggaatctataagaatgcaaaggttgcattctgcatccacaacatttcttatcaaggccgatttgccccaactgacttcaaagctctcaatttacccaatgaatttctgaagtcgtttgaattttttgatgggtatgacacgcctatcacaggggacaaaatcaactggatgcaagccggaatactagagtccgacaaggttgtgaccgtgagcccatattatgctgaagagttggtttcgggtgctgaaagaggtgtggaactagaaaaaatcattcgaaaaactggagtaattggtatcctaaatggaatggataaacaagagtgggatccttccagtgacaagttcatcaattacaaatatgatgtttcaactgtgatggaggcaaagccactcataaaggaatcccttcaagccgagttgggcttgcccgttgacaggaacattccacttataggattcattggaaggttggaagagcaaaagggttcggatattcttgttgaatccatcccagaatttatcaatgagaatgtccaaattgttatcttggggactggaaagaagcgatttgagaaacaaatcgaagagctcgaggccctttatcccgacaaggctagaggagtagcagcattcaacccatccatggctcacaagatcaatgctggtgttgactttatgttaatccctagtcggtttgagccatgtgggcttacccaactacacgctatgctctatggaacggttccaatttgtgcttcaactggtgggcttgtggattgtgtaaaagaaggcgtcacagggtttcacatgggcaagttcagcgccgaggagccagcaaagaaatgggaggagatgctactagggttacaggtggcggggagcggacctgggattgagggggaagaaatcgccccgcttgcagtggaaaatgttgctgctccttga

(19) United States
(12) Patent Application Publication
(10) Pub. No.: US 2024/0122138 A1
(43) Pub. Date: Apr. 18, 2024

(54) FUNCTION-DEFICIENT GRANULE BOUND STARCH SYNTHASE GENE AND FAGOPYRUM PLANT INCLUDING SAME

(71) Applicants: Kyoto University, Kyoto (JP); Nissey Delica Corp., Tokyo (JP)

(72) Inventors: Yasuo YASUI, Kyoto (JP); Mariko UENO, Kyoto (JP); Yasumasa KAWATE, Tokyo (JP)

(21) Appl. No.: 18/547,424

(22) PCT Filed: Feb. 21, 2022
(86) PCT No.: PCT/JP2022/006819

§ 371 Date: Aug. 22, 2023

(30) Foreign Application Priority Data
Feb. 22, 2021 (JP) 2021-026491

(51) Int. Cl. A01H 1/06; A01H 1/00; C12N 9/10; C12N 15/01

(52) U.S. Cl. CPC A01H 1/06; A01H 1/102; C12N 9/1051; C12N 15/01; C12Y 204/01242

(57) ABSTRACT

Provided is loss-of-function granule bound starch synthase genes and a Fagopyrum plant using the same. Loss-of-function granule bound starch synthase genes have been produced by: identifying granule bound starch synthase genes highly expressed in endosperm of a Fagopyrum plant from among granule bound starch synthase genes identified from mapping results of a genomic sequence of the Fagopyrum plant; and allowing the granule bound starch synthase genes thus identified to lose a function by mutagenesis, and a Fagopyrum plant has been produced which is a double-recessive homozygote homozygously having the loss-of-function granule bound starch synthase genes.

### TECHNICAL FIELD

The present invention relates to loss-of-function granule bound starch synthase genes and a Fagopyrum plant using the same, particularly, relates to loss-of-function granule bound starch synthase genes that have enabled production of a Fagopyrum plant having a glutinous trait (glutinous buckwheat), and a Fagopyrum plant using the same.

### BACKGROUND ART

Amylose and amylopectin are important substances that have large influence on the starch characteristics of cereals. Granule bound starch synthase (GBSS) synthesizes amylose with ADP glucose as a substrate. In crops of the family Poaceae or the like, amylose is not synthesized when a function of GBSS is lost. Therefore, glutinous starch consisting of only amylopectin is accumulated in endosperm.

Starch in which amylose is contained is stained bluish purple with an iodine solution, and starch is stained “reddish purple” when no amylose is contained therein. Hence, in general, screening for an individual in which glutinous starch is accumulated in endosperm is conveniently performed by using an iodine solution.

In order to produce a Fagopyrum plant having a glutinous trait, use of loss-of-function genes has received attention. In other plants except for Fagopyrum plants, attempts to improve their characteristics using loss-of-function genes are known in Patent Literature 1, Patent Literature 2, etc.

However, glutinous individuals using loss-of-function genes have not yet been confirmed for Fagopyrum plants.

### CITATION LIST

### Patent Literature

Patent Literature 1: JP-A-2016-86761
Patent Literature 2: JP-A-2018-186835

### SUMMARY OF INVENTION

### Technical Problem

An object of the present invention is to provide loss-of-function granule bound starch synthase genes and a Fagopyrum plant using the same.

The present inventors have discovered five GBSS genes from genome mapping results of buckwheat. Further, it has been found by transcriptome analysis based on RNAseq that FeGBSS1 protein and FeGBSS2 protein are highly expressed in endosperm.

Accordingly, FeGBSS1-wx1 and FeGBSS2-wx1 encoding the loss-of-function FeGBSS1 protein and the loss-of-function FeGBSS2 protein have respectively been developed using EMS. Further, an individual homozygously having FeGBSS1-wx1 and FeGBSS2-wx1 at the same time (double-recessive homozygote) has been prepared by mating. The endosperm of the double-recessive homozygote, when stained with an iodine solution, has exhibited reddish purple color, demonstrating that this double-recessive homozygote is a Fagopyrum plant having a glutinous trait (glutinous buckwheat).

### Solution to Problem

A feature of the invention of claim 1 is loss-of-function granule bound starch synthase genes produced by: identifying two granule bound starch synthase genes highly expressed in endosperm of a Fagopyrum plant from among five granule bound starch synthase genes identified from mapping results of a genomic sequence of the Fagopyrum plant; and allowing the two granule bound starch synthase genes thus identified to lose a function by mutagenesis.

A feature of the invention of claim 2 is that in the invention described in claim 1, the five granule bound starch synthase genes are identified on the basis of a query sequence of a granule bound starch synthase gene determined in advance by targeting the genomic sequence of the Fagopyrum plant.

A feature of the invention of claim 3 is that in the invention described in claim 2, the five granule bound starch synthase genes are
FesPL4_sc0224.1.g147573.t3,
FesPL4_sc0077.1.g193457.t1,
FesPL4 sc0224.1.g149056.t1,
FesPL4_sc0065.1.g82828.t1, and
FesPL4_sc0217.1.g44280.t1.

A feature of the invention of claim 4 is that in the invention described in any one of claims 1 to 3, the two granule bound starch synthase genes are identified from the numbers of reads based on transcriptome analysis for the five granule bound starch synthase genes.

A feature of the invention of claim 5 is that in the invention described in claim 4, the two granule bound starch synthase genes are FesPL4_sc0224.1.g147573.t3 and FesPL4_sc0217.1.g44280.t1.

A feature of the invention of claim 6 is that in the invention described in any one of claims 1 to 5, the mutagenesis is performed by allowing the two granule bound starch synthase genes to lose a function by ethyl methanesulfonate treatment to produce loss-of-function FeGBSS1-wx1 gene and FeGBSS2-wx1 gene.

A feature of the invention of claim 7 is that in the invention described in claim 6, the FeGBSS1-wx1 gene is a gene in which a base at position 522 of a cDNA sequence has been altered from G to A, and an amino acid at position 174 encoded by the FeGBSS1-wx1 gene has been mutated from Trp (whose codon is TGG) to a stop codon (TGA), and the FeGBSS2-wx1 gene is a gene in which a base at position 513 of a cDNA sequence has been altered from G to A, and an amino acid at position 171 encoded by the FeGBSS2-wx1 gene has been mutated from Trp (whose codon is TGG) to a stop codon (TGA).

A feature of the invention of claim 8 is a Fagopyrum plant which is a double-recessive homozygote homozygously having loss-of-function granule bound starch synthase genes produced by: identifying granule bound starch synthase genes highly expressed in endosperm of a Fagopyrum plant from among granule bound starch synthase genes identified from mapping results of a genomic sequence of the Fagopyrum plant; and allowing the granule bound starch synthase genes thus identified to lose a function by mutagenesis.

### Advantageous Effects of Invention

According to the present invention, a Fagopyrum plant can be obtained which is a double-recessive homozygote homozygously having loss-of-function granule bound starch synthase genes produced by: identifying granule bound starch synthase genes highly expressed in endosperm of a Fagopyrum plant from among granule bound starch synthase genes identified from mapping results of a genomic sequence of the Fagopyrum plant; and allowing the granule bound starch synthase genes thus identified to lose a function by mutagenesis. Therefore, the present invention is advantageously effective in providing a Fagopyrum plant having a glutinous trait and little degradation of noodle, without the addition of other ingredients.

### BRIEF DESCRIPTION OF DRAWINGS

FIG. 1 FIG. 1 is a diagram showing the nucleotide sequence of FeGBSS1_cDNA (normal) which is a granule bound starch synthase gene.

FIG. 2 FIG. 2 is a diagram showing the nucleotide sequence of FeGBSS1-wx1 cDNA (mutant) which is the loss-of-function granule bound starch synthase gene according to the present invention.

FIG. 3 FIG. 3 is a diagram showing the nucleotide sequence of FeGBSS2 cDNA (normal) which is a granule bound starch synthase gene.

FIG. 4 FIG. 4 is a diagram showing the nucleotide sequence of FeGBSS2-wx1_cDNA (mutant) which is the loss-of-function granule bound starch synthase gene according to the present invention.

FIG. 5 FIG. 5 is a photograph showing screening for an individual in which glutinous starch is accumulated in endosperm using an iodine solution as to the Fagopyrum plant according to the present invention.

### DESCRIPTION OF EMBODIMENTS

Hereinafter, Examples for carrying out the present invention will be described in detail with reference to the attached drawings.

In Examples of the present invention, nuclear DNA of Buckwheat Norin-PL1 was extracted in accordance with the approach of Yasui et al. (2016a) (Draft genome sequence of an inbred line of Chenopodium quinoa, an allotetraploid crop with great environmental adaptability and outstanding nutritional properties, DNA Research, 23, 535-546), and DNA sequences were assembled by the DenovoMAGIC method and used as the genomic sequence of buckwheat. The assembly by the DenovoMAGIC method was outsourced to NRGene Ltd. In the subcontractor Kazusa DNA Research Institute, all genes on the genomic sequence of buckwheat were predicted.

Identification of GBSS genes TBLASTX analysis targeting the genomic sequence of buckwheat was carried out using a GBSS gene (sc0002521) detected by Yasui et al. (2016b) as a query sequence. As a result, the following five GBSS genes were found to reside therein.
FesPL4 sc0224.1.g147573.t3
FesPL4_sc0077.1.g193457.t1
FesPL4_sc0224.1.g149056.t1
FesPL4_sc0065.1.g82828.t1
FesPL4 sc0217.1.g44280.t1

Hereinafter, the results (E values) of the TBLASTX analysis will be described.

| Gene name | E value |
|---|---|
| FesPL4_sc0224.1.g147573.t3 | 0.0 |
| FesPL4_sc0077.1.g193457.t1 | 0.0 |
| FesPL4_sc0224.1.g149056.t1 | 5e−180 |
| FesPL4_sc0065.1.g82828.t1 | 1e−161 |
| FesPL4_sc0217.1.g44280.t1 | 1e−157 |

Transcriptome Analysis

RNA was extracted from an immature seed of buckwheat 12 to 14 days after mating in accordance with Yasui et al. (2012) (A program for annotating and predicting the effects of single nucleotide polymorphisms, SnpEff: SNPs in the genome of Drosophila melanogaster strain w1118; iso-2; iso-3), and a library for NGS analysis was prepared using TruSeq RNA Sample prep Kit v2 from Illumina, Inc. After obtainment of nucleotide sequence reads using Hiseq X from Illumina, Inc., the cleaning of the reads was performed using trimmomatic 0.3.2 (Bolger, A. M et al. 2014). Mapping with BWA was carried out using the five genes described above as reference sequences. The numbers of reads finally mapped to the genes of the reference sequences were regarded as expression intensity.

The results are as described below, and it was found that two GBSS genes FesPL4_sc0224.1.g147573.t3 and FesPL4 sc0217.1.g44280.t1 were highly expressed in endosperm. FesPL4_sc0224.1.g147573.t3 having a higher expression level was designated as FeGBSS1, and FesPL4_sc0217.1.g44280.t1 was designated as FeGBSS2.

| Predicted gene | The numbers of reads |
|---|---|
| FesPL4_sc0224.1.g147573.t3 | 7,295,100 |
| FesPL4_sc0077.1.g193457.t1 | 1,342 |
| FesPL4_sc0224.1.g149056.t1 | 754 |
| FesPL4_sc0065.1.g82828.t1 | 11,395 |
| FesPL4_sc0217.1.g44280.t1 | 6,210,988 |

Induction of Mutation and Mass Breeding

In order to allow FeGBSS1 and FeGBSS2 to lose a function, mutagenesis with EMS was carried out. Approximately 4,600 seeds were treated with a 0.33% EMS solution for 15 hours and cultivated in Kyoto University Experimental Farm. Three to six seeds were harvested from one individual to obtain 7,490 seeds. The seeds were further treated with 0.6% EMS for 15 hours, and seeds were finally harvested from 2,710 individuals. For seed harvesting, one or two seeds were harvested per individual. The seeds were inoculated and cultivated in Kyoto University Experimental Farm, and seeds were harvested from 3,336 individuals on an individual basis. At the time of cultivation, the leaves of 12 individuals were collectively subjected in equal amounts to DNA extraction to prepare 278 DNA bulks (12 individuals×278 bulks=3,336).

Development of FeGBSS1-Wx1 and FeGBSS2-Wx1 Using Mutation Detection

PCR primers for FeGBSS1 and FeGBSS2 were designed, and PCR amplification was performed with the 278 DNA bulks as templates under the following conditions.

98° C. for 2 min, (98° C. for 10 sec, 58° C. for 5 sec, and 72° C. for 90 sec)×30 cycles, and 72° C. for 5 sec PCR primers were as follows.

Primer for FeGBSS1 amplification
GBSS_sc0005258_Fw1;
GACGTCACTCACAATCACAAGTAGC
Primer for FeGBSS2 amplification
GBSS_sc0002521_Fw3;
AATACCGCTGTGTGTATGGCAAG
GBSS_sc0002521_Rv1205;
GGTCCTGAGAAAAATTTGTTGTTG

After PCR, the PCR products were prepared into libraries on a bulk basis using Nextera XT DNA Library Prep kit (Illumina, Inc.), and nucleotide sequence reads were obtained using Hiseq X (Illumina, Inc.). Then, the cleaning of the reads using trimmomatic 0.3.2 and mapping to the reference sequences (FeGBSS1 and FeGBSS2 gene sequences) with BWA (Li and Durbin, 2009) (Draft genome sequence of an inbred line of Chenopodium quinoa, an allotetraploid crop with great environmental adaptability and outstanding nutritional properties, DNA Research, 23, 535-546) were performed.

Then, treatment (BAM conversion, sorting, and mpileup) with samtools (Li et al., 2009) (The Sequence Alignment/Map format and SAMtools, Bioinformatics, 25, 2078-2079), Vcf preparation with VarScan (Koboldt et al., 2009) (VarScan: variant detection in massively parallel sequencing of individual and pooled samples. Bioinformatics (Oxford, England), 25, 2283-2285 PMID: 19542151), and mutation detection with SnpEff (Cingolani et al., 2012) (A program for annotating and predicting the effects of single nucleotide polymorphisms, SnpEff: SNPs in the genome of Drosophila melanogaster strain w1118; iso-2; iso-3) were performed.

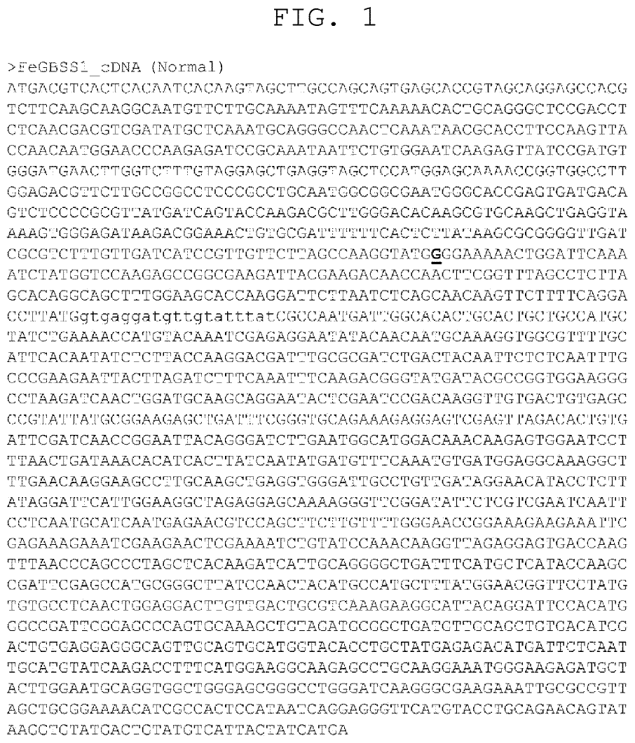

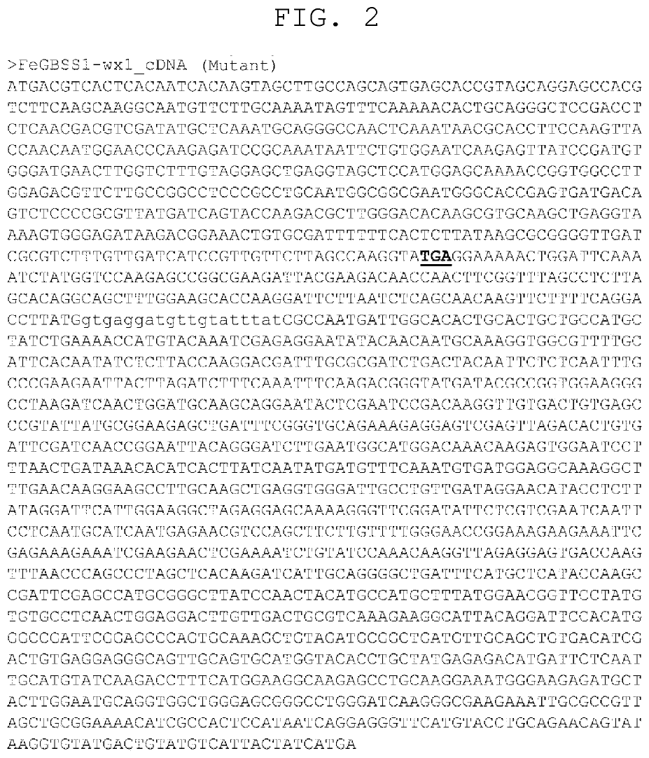

As a result, as shown in FIG. 1, FeGBSS1-wx1 gene was developed in which a base at position 522 of the cDNA sequence of FeGBSS1 was altered from G to A. As shown in FIG. 2, an amino acid at position 174 encoded by the FeGBSS1-wx1 gene was mutated from Trp (whose codon is TGG) to a stop codon (TGA), and the FeGBSS1 protein was considered to lose a function.

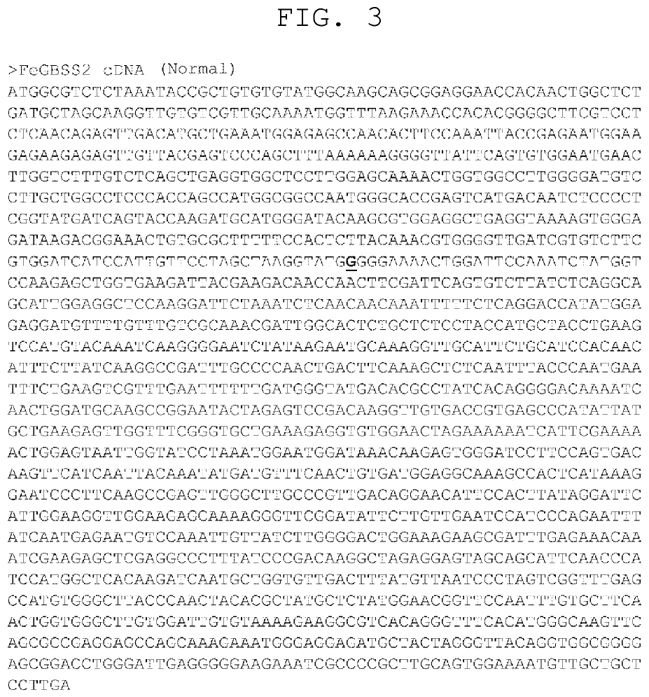

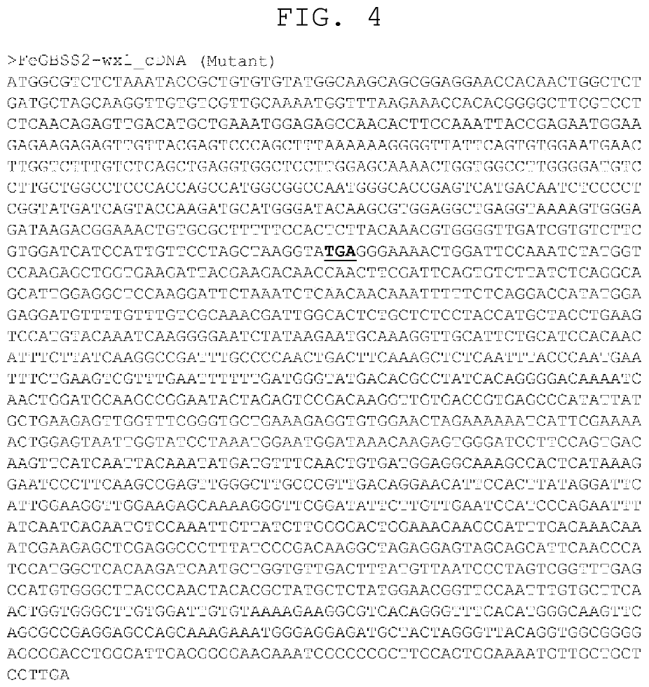

As shown in FIG. 3, FeGBSS2-wx1 gene was developed in which a base at position 513 of the cDNA sequence of FeGBSS2 was altered from G to A. As shown in FIG. 4, an amino acid at position 171 encoded by the FeGBSS2-wx1 gene was mutated from Trp (whose codon is TGG) to a stop codon (TGA), and the function of the FeGBSS2 protein was considered to be lost.

Development of glutinous buckwheat by mating Two genes of FeGBSS1 and FeGBSS2 have a high expression level and require bleeding an individual homozygously retaining FeGBSS1-wx1 and FeGBSS2-wx1 at the same time (double-recessive homozygote) for imparting a glutinous trait to buckwheat. Accordingly, first, double heterozygotes (genotype; FeGBSS1/FeGBSS1-wx1 and FeGBSS2/FeGBSS2-wx1) were bred, and a glutinous buckwheat individual (genotype; FeGBSS1-wx1/FeGBSS1-wx1 and FeGBSS2-wx1/FeGBSS2-wx1) was bred by the mating of the double heterozygotes. In this respect, for the determination of the genotypes, the FeGBSS1 and FeGBSS2 genes were amplified by PCR, and their nucleotide sequences were determined by the Sanger method. The PCR conditions were as mentioned above, and the following PCR primers were used in the determination of the nucleotide sequences by the Sanger method.

Sequencing primer for GBSS1-wx1 detection,

AGCTGAGGTAAAAGTGGGAGATAAG

Sequencing primer for GBSS2-wx1 detection,

GGGGTTGATCGTGTCTTCGT

Staining with Iodine Liquid

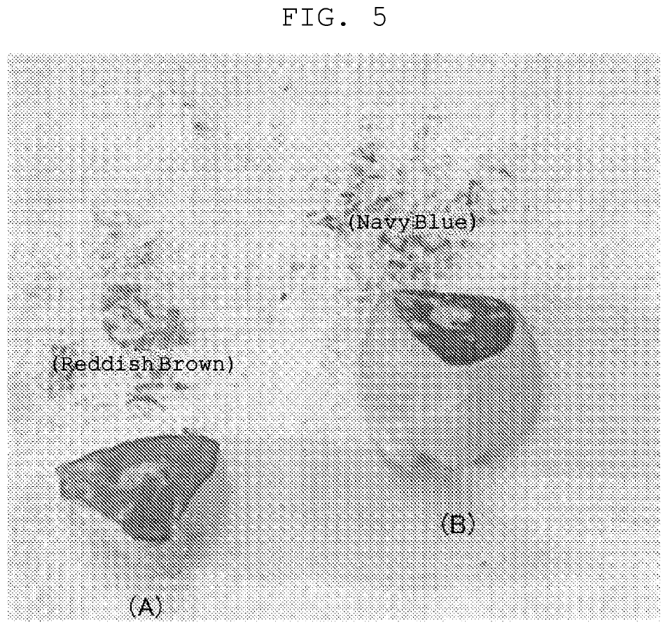

The seed of buckwheat was cut, and an iodine liquid (0.37 g of purified iodine and 0.74 g of KI were dissolved in 400 ml of distilled water) was added dropwise to the cut surface to stain starch in buckwheat. As a result, glutinous starch was stained reddish brown as shown in FIG. 5 (A), and starch of usual buckwheat used as a control was stained navy blue as shown in FIG. 5 (B).

An amylose content in the endosperm of the Fagopyrum plant according to the present invention is considered to be 20% or less of total starch.

The present invention is not limited by Examples mentioned above, and many changes or modifications can be made in the present invention by the ordinary creativity of those skilled in the art without departing from the technical brief of the present invention.

1. Loss-of-function granule bound starch synthase genes produced by:
identifying two granule bound starch synthase genes highly expressed in endosperm of a Fagopyrum plant from among five granule bound starch synthase genes identified from mapping results of a genomic sequence of the Fagopyrum plant; and
allowing the two granule bound starch synthase genes thus identified to lose a function by mutagenesis.

2. The loss-of-function granule bound starch synthase genes according to claim 1, wherein
the five granule bound starch synthase genes are
identified on the basis of a query sequence of a granule bound starch synthase gene determined in advance by targeting the genomic sequence of the Fagopyrum plant.

3. The loss-of-function granule bound starch synthase genes according to claim 2, wherein
the five granule bound starch synthase genes are
FesPL4_sc0224.1.g147573.t3,
FesPL4_sc0077.1.g193457.t1,
FesPL4_sc0224.1.g149056.t1,
FesPL4_sc0065.1.g82828.t1, and
FesPL4_sc0217.1.g44280.t1.

4. The loss-of-function granule bound starch synthase genes according to claim 1, wherein
the two granule bound starch synthase genes are
identified from the numbers of reads based on transcriptome analysis for the five granule bound starch synthase genes.

5. The loss-of-function granule bound starch synthase genes according to claim 4, wherein
the two granule bound starch synthase genes are
FesPL4_sc0224.1.g147573.t3 and
FesPL4_sc0217.1.g44280.t1.

6. The loss-of-function granule bound starch synthase genes according to claim 1, wherein
the mutagenesis is performed by allowing the two granule bound starch synthase genes to lose a function by ethyl methanesulfonate treatment to produce loss-of-function FeGBSS1-wx1 gene and FeGBSS2-wx1 gene.

7. The loss-of-function granule bound starch synthase genes according to claim 6, wherein
the FeGBSS1-wx1 gene is
a gene in which a base at position 522 of a cDNA sequence has been altered from G to A, and an amino acid at position 174 encoded by the FeGBSS1-wx1 gene has been mutated from Trp (whose codon is TGG) to a stop codon (TGA), and
the FeGBSS2-wx1 gene is
a gene in which a base at position 513 of a cDNA sequence has been altered from G to A, and an amino acid at position 171 encoded by the FeGBSS2-wx1 gene has been mutated from Trp (whose codon is TGG) to a stop codon (TGA).

8. A Fagopyrum plant which is a double-recessive homozygote homozygously having loss-of-function granule bound starch synthase genes produced by:
identifying granule bound starch synthase genes highly expressed in endosperm of a Fagopyrum plant from among granule bound starch synthase genes identified from mapping results of a genomic sequence of the Fagopyrum plant; and
allowing the granule bound starch synthase genes thus identified to lose a function by mutagenesis.
